(19) United States
(12) Patent Application Publication
(10) Pub. No.: US 2007/0092473 A1
(43) Pub. Date: Apr. 26, 2007

(54) Cosmetic composition containing a statistical polymer with a linear main chain of ethylenic nature

(75) Inventors: Xavier Schultze, Paris (FR); Marco Vicic, Bry Sur Marne (FR)

Correspondence Address: OLIFF & BERRIDGE, PLC, P.O. BOX 19928, ALEXANDRIA, VA, 22320, US

(73) Assignee: L'OREAL, PARIS (FR)

(21) Appl. No.: 11/583,768

(22) Filed: Oct. 20, 2006

Related U.S. Application Data
(60) Provisional application No. 60/740,285, filed on Nov. 29, 2005.

(30) Foreign Application Priority Data
Oct. 21, 2005 (FR) 05 53215

(51) Int. Cl. A61K 8/81

(52) U.S. Cl. 424/70.16

(57) ABSTRACT

A cosmetic composition includes, in a physiologically acceptable medium, at least one statistical copolymer with a linear main chain of ethylenic nature, in which the copolymer: (i) has a weight-average molecular mass of between 15,000 and 600,000 g/mol, and has: (a) at least 70% by weight of “monomer units” derived from monomers for which the homopolymers are hydrophobic and have a glass transition temperature of greater than 40° C., (b) at least one “monomer unit” derived from an at least partially neutralized ionic hydrophilic monomer, (c) 0 to 25% by weight of “monomer units” derived from additional monomers for which the homopolymers have a glass transition temperature of less than 40° C., chosen from: the acrylates of formula:
CH₂═CHCOOR₁₂
in which R₁₂ represents a linear or branched C₁ to C₁₂ alkyl group with the exception of a tert-butyl group, in which is (are) optionally intercalated one or more identical or different heteroatoms, the alkyl group also possibly being optionally substituted with one or more identical or different substituents chosen from hydroxyl groups and halogen atoms, and the methacrylates of formula:
CH₂═C(CH₃)COOR₁₄
in which R₁₄ represents a linear C₄ to C₁₂ alkyl group or a branched C₅ to C₁₂ alkyl group, and (ii) has an overall glass transition temperature of greater than or equal to 45° C.

This non provisional application claims the benefit of French Application No. 05 53215 filed on Oct. 21, 2005 and U.S. Provisional Application No. 60/740,285 filed on Nov. 29, 2005.

### BACKGROUND

The present invention relates to an anti-wrinkle composition containing at least one statistical copolymer with a linear main chain of ethylenic nature, and to the use of this copolymer as a tensioning agent in a cosmetic composition, intended especially for treating, reducing, effacing and/or smoothing out wrinkles and fine lines on human skin.

In the course of the ageing process, various signs appear on the skin, which are very characteristic of this ageing, which are reflected especially by a change in the structure and functions of the skin. The main clinical signs of ageing of the skin are especially the appearance of fine lines and deep wrinkles, which increase with age. Disorganization of the “grain” of the skin, i.e. the microrelief is less uniform and has an anisotropic nature, is in particular observed.

It is known practice to treat these signs of ageing by using cosmetic or dermatological compositions containing active agents capable of combating ageing, such as α-hydroxy acids, β-hydroxy acids and retinoids. These active agents act on wrinkles by removing the dead. cells from the skin and by accelerating the process of cell renewal. However, these active agents have the drawback of being effective in treating wrinkles only after a certain period of application. Now, it is increasingly sought to obtain an immediate effect of the active agents used, rapidly leading to smoothing-out of wrinkles and fine lines and to the disappearance of fatigue marks.

A subject of the present invention is, precisely, the use of a particular copolymer that allows this effect to be obtained immediately.

It is known practice to use certain tensioning agents of natural origin, such as proteins and protein hydrolysates of plant origin, as is described especially in WO 98/29091, or alternatively a plant polysaccharide and hydrolysed casein as tensioning active agents, as especially described in WO 96/19180. However, the use of substances of natural origin is limited for health and minimum tensioning effect reasons.

It is moreover known practice to use inorganic materials such as colloidal silica and clays as tensioning agents. The performance qualities achieved in this respect are very high. However, the tensioning effect of these inorganic materials is cancelled out in emulsion in the presence of glycerin.

Other compositions with a tensioning effect use synthetic polymers.

Thus, WO 98/29092 discloses a composition with a tensioning effect comprising an aqueous dispersion of a polymeric system containing at least one polymer of synthetic origin with a molecular weight of greater than 670 000 g/mol, chosen from various types of polyurethanes, polyureas, acrylic polymers or copolymers and sulfonated isophthalic acid polymers, and mixtures thereof. The cosmetic feel of these compositions is, however, not always satisfactory.

EP 1,038,519 also discloses the use of certain specific silicone polymers for their role as agents with a tensioning effect, and WO 00/30595 discloses copolymers containing, by weight, from 20% to 90% of a vinyl lactam, from 1% to 55% of a polymerizable carboxylic acid and from 1% to 25% of a hydrophobic monomer such as a C₁₀ to C₂₄ alkyl acrylate or methacrylate, for providing soft gels that may be used in anti-wrinkle cosmetic compositions.

FR 2,843,025 describes the use of interpenetrated polymer networks as agents for smoothing out wrinkles and fme lines and/or for retensioning the skin, and FR 2,822,676 describes a film-forming cosmetic composition comprising, as film-forming agent, at least one acrylic copolymer in a high content, i.e. between 20% and 50% by weight relative to the weight of the composition.

### SUMMARY OF THE INVENTION

There is still a need for compounds that offer an immediate, sufficient and long-lasting tensioning effect, without any risk to the consumer.

In addition, there is a need for synthetic polymeric materials that have a high tensioning effect while at the same time being able to be formulated in the presence of glycerin.

There is also a need for synthetic polymeric materials that can be conveyed in an aqueous medium and that have very high rigidity after application to the skin and after evaporation of the volatile materials.

One subject of the present invention is thus a cosmetic composition comprising, in a physiologically acceptable medium, at least one statistical copolyrner with a linear main chain of ethylenic nature, in which the copolymer:
(i) has a weight-average molecular mass of between 15,000 and 600,000 g/mol, and consists of:
(a) at least 70% by weight of “monomer units” derived from monomers for which the homopolymers are hydrophobic and have a glass transition temperature of greater than 40° C.,
(b) at least one “monomer unit” derived from an at least partially neutralized ionic hydrophilic monomer, and
(c) 0 to 25% by weight of “monomer units” derived from additional monomers for which the homopolymers have a glass transition temperature of less than 40° C., chosen from:
   the acrylates of formula:
      CH₂═CHCOOR₁₂
      in which R₁₂ represents a linear or branched C₁ to C₁₂ alkyl group (with the exception of a tert-butyl group), in which is (are) optionally intercalated one or more identical or different heteroatoms, the alkyl group also possibly being optionally substituted with one or more identical or different substituents chosen from hydroxyl groups and halogen atoms, and
   the methacrylates of formula:
      CH₂═C(CH₃)COOR₁₄
      in which R₁₄ represents a linear C₄ to C₁₂ alkyl group or a branched C₅ to C₁₂ alkyl group, and
(ii) has an overall glass transition temperature of greater than or equal to 45° C.

A subject of the invention is also the cosmetic method for tensioning the skin comprising the application of a cosmetic composition containing a copolymer as above mentioned.

### DETAILED DESCRIPTION OF EMBODIMENTS

In the context of this description and of the attached claims, the term “tensioning agent” means compounds capable of having an apparent tensioning effect, i.e. of smoothing out the skin and of immediately reducing, or even making disappear, the wrinkles and fine lines.

More specifically, it may be considered that a composition has a tensioning effect when, in the test presented in Example 10-1, the value of the tensioning effect TE is greater than 60%.

The composition used in the present invention contains, in addition to the abovementioned copolymer, a physiologically acceptable medium, e.g., a medium that is compatible with the skin and its integuments, mucous membranes and semi-mucous membranes.

The present invention also relates to a cosmetic method for reducing or even treating age signs and especially skin wrinkles comprising the application to the said skin of at least one composition comprising at least one statistical copolymer with a linear main chain of ethylenic nature as defined above, in an amount that is effective for smoothing out or even effacing the wrinkles and fine lines on human skin by means of a tensioning effect.

The present invention further relates to a cosmetic method for treating aged and especially wrinkled skin, comprising the application to the said skin of at least one composition comprising at least one statistical copolymer with a linear main chain of ethylenic nature as defined above, in an amount that is effective for effacing the wrinkles by means of a tensioning effect.

In the context of the present invention, the term “of ethylenic nature” qualifies polymers comprising a main chain comprising only monomer units, 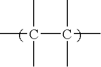
the side chains possibly consisting of carbon, oxygen, nitrogen, hydrogen, sulphur and/or phosphorus atoms.

It is noted in particular that the presence of silicon atoms in the side chain is excluded from the scope of the invention.

The term “monomer unit” denotes the largest constituent unit of the structure of a macromolecule formed from the same monomer molecule.

In the context of the present invention, the term “alkyl” means a linear or branched, saturated or unsaturated, cyclic or non-cyclic hydrocarbon-based chain. Among the alkyl groups that are suitable for use in the invention, mention may be made especially of methyl, ethyl, isopropyl, n-propyl, n-butyl, t-butyl, —CH₂-t-butyl, pentyl, n-hexyl, cyclopropyl, cyclopentyl, cyclohexyl, cyclohexylmethyl, heptyl, octyl, nonyl, decyl, norbornyl and adamantyl groups.

In the context of the present invention, the term “aryl” means a monocyclic or bicyclic system containing one or two aromatic nuclei. Among the aryl groups, mention may be made of phenyl, naphthyl, tetrahydronaphthyl and indanyl.

The term “aralkyl” means an aryl group linked to an alkyl group, such as a benzyl group.

The term “heterocyclic group” means a 4- to 12-membered ring containing one or more identical or different heteroatoms chosen from O, N, S and P. The said heterocyclic group may or may not comprise double bonds. This term also comprises bicyclic groups in which a 3-, 4-, 5- or 6-membered heterocycle is fused to a phenyl group or to a cycloalkyl such as cyclohexane, or alternatively to another heterocycle.

Among these heterocyclic groups, mention may be made of indolyl, quinolyl, isoquinolyl, tetrahydroquinolyl, benzofuryl and benzothienyl. The term “heterocyclic group” especially covers pyrrolyl, pyrrolinyl, pyrrolidinyl, pyrazolyl, pyrazolinyl, pyrazolidinyl, imidazolyl, imidazolinyl, imidazolidinyl, pyridyl, piperidyl, pyrazinyl, piperazinyl, pyrimidinyl, pyridazinyl, oxazolyl, oxazolidinyl, isoxazolyl, isoxazolidinyl, morpholinyl, thiazolyl, thiazolidinyl, isothiazolyl, isothiazolidinyl, indolyl, quinolinyl, isoquinolinyl, benzimidazolyl, benzothiazolyl, benzoxazolyl, furyl and thienyl.

The term “heterocyclylalkyl” means a heterocyclic group linked to an alkyl group.

The halogen atoms comprise chlorine, bromine, iodine and fluorine.

Unless otherwise mentioned, the heteroatoms comprise oxygen, nitrogen, sulphur and phosphorus atoms.

The term “between . . . and . . . ” means that the limits are also included.

Copolymers

The copolymer present in the composition according to the present invention is a statistical copolymer with a linear main chain of ethylenic nature.

The composition may also comprise a mixture of such copolymers.

The copolymer that is useful in the context of the present invention has a weight-average molecular mass of between 15,000 and 600,000 g/mol. The molecular mass is preferably between 20,000 and 200,000 g/mol and even more preferably ranges from 55,000 to 200,000 g/mol. The term “weight-average molecular mass” means the molecular-mass Mw at the peak of the distribution curve.

Moreover, its overall glass transition temperature is greater than or equal to 45° C., especially ranging from 45° C. to 300° C.

The term “glass transition temperature”, the abbreviation of which is Tg, means the temperature below which the polymer is rigid. When the temperature increases, the polymer passes through a transition state that allows the macromolecular chains to slide relative to each other and the polymer softens.

The term “overall glass transition temperature” is used to indicate that the copolymer may include different monomers, the respective homopolymers of which may have different glass transition temperatures, and that it is the copolymer per se that has the said overall glass transition temperature.

Thus, in the context of the present invention, it is preferred to use copolymers with an overall glass transition temperature of greater than 60° C., especially greater than 60° C. and less than 300° C.

In the context of the present invention, the protocol for measuring the glass transition temperatures of the copolymers or homopolymers formed by the monomers that are useful for preparing copolymers uses a characterization by DSC (Differential Scanning Calorimetry) and is detailed below:

The transitions of the film (glass transitions, melting, etc.) are studied by DSC on the basis of 2 cycles of heating/cooling at 10° C./minute between −140° C. and 130° C. (approximately 2 hours). The measurements are performed under a flush of nitrogen and using hermetic crucibles so as not to modify the composition of the film, by vaporization of the solvent, during the DSC study. The polymer film is prepared by drying the aqueous solution directly deposited (40 μl) in the thermal analysis crucibles. The drying of the solution takes place under controlled conditions over 48 hours at room temperature and at 50±5% relative humidity.
Apparatus: DSC 2920 from TA Instruments
Purge gas: Alphagaz 2 nitrogen at 50 ml/minute
Crucible: 50 μl crimped stainless-steel crucible from Perkin Elmer
Energy and temperature calibration: fusion of indium
Specimen: conditioned by drying (about 10 mg)
Heat treatments:
1. CO: cooling from +25° C. to −140° C. at 10° C./minute
2. COa: equilibration at −140° C.
3. H1: heating from −140° C. to +130° C. at 10° C./minute
4. C1: cooling from +130C to −140° C. at 10° C./minute
5. C1a: equilibration at −140° C.
6. H2: heating from −140° C. to +130° C. at 10° C./minute

Two samples are studied for each product.

The copolymer is preferably in dispersion in a polar liquid, the said polar liquid preferably being water.

When the copolymer is in aqueous dispersion, its content in the said dispersion may range from 0.1% to 30% by weight and preferably from 5% to 20% by weight, expressed as dry matter, relative to the total weight of the said dispersion. As is illustrated in the examples, the copolymer may typically be dispersed at 7% by weight in water and give rise to a tensioning effect of greater than 70% in the retraction test outlined in the examples.

The structure of the copolymer is detailed in the description that follows.

The copolymer comprises at least 70% by weight, relative to its total weight, of “monomer units” derived from monomers whose homopolymers have a glass transition temperature of greater than 40° C. These “monomer units” may be of the same nature or of different nature. In other words, the copolymer may comprise only one type of “monomer units” whose homopolymer has a glass transition temperature of greater than 40° C., or alternatively “monomer units” of different nature, it being understood that the corresponding monomers are all in accordance with the requirement recalled above concerning the glass transition temperature.

In particular, the copolymer according to the invention comprises more than 72%, especially more than 75% and preferably more than 80% by weight, and especially up to 95% by weight, relative to its total weight, of “monomer units” derived from monomers whose homopolymers have a glass transition temperature of greater than 40° C.

The copolymer also comprises at least one “monomer unit” derived from an ionic hydrophilic monomer, and also from 0 to 25% by weight of “monomer units” derived from additional monomers whose homopolymers have a glass transition temperature of less than 40° C.

Hydrophobic Monomers Whose Homopolymers Have a Glass Transition Temperature of Greater than 40° C.

For the purposes of the present invention, the term “hydrophobic monomer” means a monomer whose homopolymer is insoluble in water at a concentration of greater than 5% by weight, at 25° C., and which does not form either in water, under these conditions, a stable dispersion or suspension of fme, generally spherical, particles with a mean particle size of less than 1 μm, and more generally between 5 and 400 nm, or even between 10 and 250 nm, as measured by light scattering.

Among the monomers from which the hydrophobic “monomer units” mentioned above are derived, those whose homopolymers have a glass transition temperature of greater than or equal to 60° C. and especially less than or equal to 300° C. are preferred.

The monomers that are useful for the preparation of the copolymers included in the compositions according to the present invention and whose homopolymers have glass transition temperatures of greater than 40° C. are preferably chosen from vinyl compounds, acrylates, methacrylates and (meth)acrylamides, and in particular from the following monomers:
the vinyl compounds of formula:
   CH₂═CHR₁,
   in which R₁ is a group: 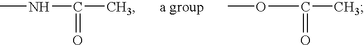
   a C₃ to C₈ cycloalkyl group; a C₆ to C₂₀ aryl group; a C₇ to C₃₀ aralkyl group (C₁ to C₄ alkyl group); a heterocyclic group; a heterocyclylalkyl group (C₁ to C₄ alkyl) such as a furfuryl group; the said cycloalkyl, aryl, aralkyl, heterocyclic or heterocyclylalkyl groups possibly being substituted with one or more identical or different substituents chosen from hydroxyl groups, halogen atoms and linear or branched C₁ to C₄ alkyl groups in which is (are) optionally intercalated one or more identical or different heteroatoms, and the said alkyl groups also possibly being substituted with one or more substituents chosen from hydroxyl groups and halogen atoms.

According to one preferred variant, R₁ is a group 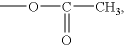
a C₃ to C₈ cycloalkyl group or a C₆ to C₂₀ aryl group. Particularly preferred examples of vinyl monomers whose homopolymers have a glass transition temperature of greater than 40° C. are vinylcyclohexane, styrene and vinyl acetate.
The acrylates of formula:
   CH₂═CHCOOR₂
   in which R₂ is a tert-butyl group; a C₃ to C₈ cycloalkyl group optionally bridged with a C₁ to C₄ alkylene group optionally substituted with one or more C₁ to C₄ alkyl groups; a C₆ to C₂₀ aryl group; a C₇ to C₃₀ aralkyl group (C₁ to C₄ alkyl group); a heterocyclic group; a heterocyclylalkyl group (C₁ to C₄ alkyl); the said cycloalkyl, aryl, aralkyl, heterocyclic or heterocyclylalkyl groups possibly being substituted with one or more identical or different substituents chosen from hydroxyl groups, halogen atoms and linear or branched C₁ to C₄ alkyl groups in which is (are) optionally intercalated one or more identical or different heteroatoms, the said alkyl groups also possibly being substituted with one or more identical or different substituents chosen from hydroxyl groups and halogen atoms.

According to one preferred variant, R₂ is a tert-butyl group, a C₃ to C₈ cycloalkyl group optionally bridged with a C₁ to C₄ alkylene group, or a C₇ to C₃₀ aralkyl group (C₁ to C₄ alkyl group).

Particularly preferred examples of acrylates whose homopolymers have a glass transition temperature of greater than 40° C. are benzyl acrylate, cyclohexyl acrylate, tert-butyl acrylate, isobornyl acrylate and norbornyl acrylate.
The methacrylates of formula:
   CH₂═C(CH₃)COOR₃
   in which R₃ is an isobutyl or tert-butyl or a linear or branched C₁ to C₃ alkyl group, such as a methyl, ethyl, propyl or isobutyl group, these alkyl groups also possibly being substituted with one or more identical or different substituents chosen from hydroxyl groups and halogen atoms; a C₃ to C₈ cycloalkyl group optionally bridged with a C₁ to C₄ alkylene group optionally substituted with one or more C₁ to C₄ alkyl groups; a C₆ to C₂₀ aryl group; a C₇ to C₃₀ aralkyl group (C₁ to C₄ alkyl group); a heterocyclic group; a heterocyclylalkyl group (C₁ to C₄ alkyl); the said cycloalkyl, aryl, aralkyl, heterocyclic or heterocyclylalkyl groups possibly being substituted with one or more identical or different substituents chosen from hydroxyl groups, halogen atoms and linear or branched C₁ to C₄ alkyl groups in which is (are) optionally intercalated one or more identical or different heteroatoms, the said alkyl groups also possibly being substituted with one or more identical or different substituents chosen from hydroxyl groups and halogen atoms.

According to one preferred variant, R₃ is an isobutyl or tert-butyl group, a linear or branched C₁ to C₃ alkyl group, a C₃ to C₈ cycloalkyl group optionally bridged with a C₁ to C₄ aLkylene group, or a C₇ to C₃₀ aralkyl group (C₁ to C₄ alkyl group).

Particularly preferred examples of methacrylates whose homopolymers have a glass transition temperature of greater than 40° C. are methyl, ethyl, isobutyl, cyclohexyl, benzyl, tert-butyl, isobornyl and norbomyl methacrylate.
The (meth)acrylamides of formula: 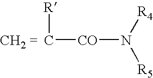
   in which R′ denotes H or —CH₃, and in which R₄ and R₅, which may be identical or different, each represent a hydrogen atom or a linear or branched linear C₄ to C₁₂ alkyl group, it being understood that R₄ and R₅ cannot simultaneously represent a hydrogen atom.

Particularly preferred examples of (meth)acrylamide monomers whose homopolymers have a glass transition temperature of greater than 40° C. are N-butyl-acrylamide, N-t-butylacrylamide and N,N-dibutylacrylamide.

According to one preferred embodiment of the invention, the monomers whose homopolymers have a glass transition temperature of greater than 40° C. are chosen from styrene, benzyl acrylate, C₃ to C₈ cycloalkyl acrylate optionally bridged with a C₁ to C₄ alkylene group, tert-butyl acrylate, (C₁-C₃)alkyl methacrylate, tert-butyl methacrylate, benzyl methacrylate and C₃ to C₈ cycloalkyl methacrylate optionally bridged with a C₁ to C₄ alkylene group.

The preferred monomers whose homopolymers have a glass transition temperature of greater than 40° C. are benzyl acrylate, cyclohexyl acrylate, tert-butyl acrylate, isobomyl. acrylate, norbomyl acrylate, methyl, ethyl, isobutyl, cyclohexyl, benzyl, tert-butyl, isobornyl or norbomyl methacrylate and styrene. They are more preferentially chosen from methyl methacrylate and cyclohexyl. methacrylate.

Ionic Hydrophilic Monomer

The copolymer present in the composition according to the present invention also comprises at least one “monomer unit” derived from an at least partially neutralized ionic hydrophilic monomer.

This ionic hydrophilic monomer may be chosen especially from anionic hydrophilic monomers, cationic hydrophilic monomers and amphoteric monomers, and mixtures thereof.

Typically, the copolymer may contain between 5% and 30% by weight of “monomer units” derived from ionic hydrophilic monomers, relative to its total weight. More particularly, the copolymer may contain between 5% and 25% by weight, especially between 5% and 20% by weight, preferably between 5% and 18% by weight relative to its total weight. The copolymer may for example contain between 10% and 25% by weight and preferably between 10% and 20% by weight and more preferably between 10% and 18% by weight of “monomer units” derived from ionic hydrophilic monomers, relative to its total weight.

In one preferred embodiment, the copolymer contains between 5% and 25% by weight, relative to its total weight of “monomer units” derived from ionic hydrophilic monomers provided that when the copolymer comprises additional monomer(s) and when at least one of said additional monomer(s) whose corresponding homopolymer has a glass transition temperature of less than 40° C., is ethylhexyl acrylate the copolymer contains between 5% and 18% by weight of “monomer units” derived from ionic hydrophilic monomers relative to its total weight.

For the purposes of the present invention, the term “partially neutralized” means a “monomer unit” with a non-zero degree of neutralization, preferably of greater than or equal to 50%.

Advantageously, all the “monomer units” have a degree of neutralization of greater than or equal to 50%, preferably greater than or equal to 70% and in particular of at least 90%, or even of 100%.

The degree of neutralization may be defined as being the ratio of the number of neutralized ionic fuictions to the initial number of ionized functions, i.e. before neutralization

Advantageously, the degree of neutralization of all the “monomer units” will be adjusted so as to allow dispersion in water of the copolymers according to the invention.

Among the anionic hydrophilic monomers are monomers of formula (I):
CH₂═CR₆ (Z)ₙ (R₇)ₘ Y (I)
in which Z takes one of the following meanings: C(═O)O, C(═O)NH, O, O(C═O) (acetates),
n is equal to 0 or 1, preferably equal to 0,
m is equal to 0 or 1, preferably equal to 0,
R₆ is a hydrogen atom, a CH₃ group or a (C₂-C₃₀)alkyl group,
R₇ is chosen from linear, saturated or unsaturated and/or branched and/or cyclic (aromatic or non-aromatic) C₁ to C₃₀ alkylene groups, optionally including one or more heteroatoms, and Y is chosen from the following groups: —COOH, —SO₃H, —OSO₃H, —OP(OH)₂, —OPO(OH)₂.

Preferentially, R₆ is a hydrogen atom or a CH₃ or C₂H₅ group, preferably CH₃.

R₇ is chosen, for example, from C₁ to C₃₀ alkylene, phenylene, benzylene, —(CH₂—CH═CH)— and —(CHOH)— groups. Preferably, R₇ is chosen from linear, branched or cyclic, C₁ to C₆ alkylene groups and phenylene and benzylene groups.

According to one particular embodiment, R₆ is a hydrogen atom or a CH₃ or a C₂H₅ group, preferably a CH₃ group, and R₇ is chosen from C₁ to C₃₀ alkylene, phenylene, benzylene, (CH₂—CH═CH)— and (CHOH) groups.

Among the anionic hydrophilic monomers, mention may be made especially of:
ethylenically unsaturated monomers comprising at least one carboxylic acid (COOH), phosphonic acid (PO₃H₂) or sulfonic acid (SO₃H) finction, for instance acrylic acid, methacrylic acid, 2-carboxyethyl (meth)acrylate, vinylbenzoic acid, acrylamidoglycolic acid CH₂═CHCONHCH(OH)CO₂H, maleic acid, acrylamidopropanesulfonic acid, acrylamido-2-methylpropanesulfonic acid, styrenesulfonic acid, vinylsulfonic acid, 3-propylsulfonic acid methacrylate or acrylate (CH₂═C(CH₃)CO₂(CH₂)₃SO₃H), 2-ethyl-sulfonic acid methacrylate or acrylate (CH₂═C(CH₃)CO₂(CH₂)₂SO₃H) and methyl vinyl sulfone, vinylphosphonic acid (CH₂═CH—PO(OH)₂) and 2-ethylphosphonic acid methacrylate (CH₂═C(CH₃)COOCH₂CH₂OP(O)(OH)₂),
carboxylic anhydrides bearing a vinyl bond, such as maleic anhydride,
diacids such as crotonic acid, itaconic acid, fumaric acid or maleic acid,
and mixtures thereof.

Preferably, the anionic hydrophilic monomer is (meth)acrylic acid.

Neutralization of the anionic groups may be performed with a mineral base, such as LiOH, NaOH, KOH, Ca(OH)₂, NH₄OH or Zn(OH)₂; or with an organic base such as a primary, secondary or tertiary alkylamine, especially triethylamine or butylamine. This primary, secondary or tertiary alkylamine may comprise one or more nitrogen and/or oxygen atoms and may thus comprise, for example, one or more alcohol functions; mention may be made especially of 2-amino-2-methylpropanol, triethanolamine and 2-dimethylaminopropanol. Mention may also be made of lysine or 3-(dimethylamino)propylamine.

Among the cationic hydrophilic monomers that are included are the monomers of formula (II):
CH₂═CR₈ (Z)ₙ (Rg)ₘ X (II)
in which Z takes one of the following meanings: C(═O)O, C(═O)NH, O, O(C═O),
n is equal to 0 or 1,
m is equal to 0 or 1,
R₈ is a hydrogen atom, a CH₃ group or a (C₂-C₃₀)alkyl group,
R₉ is chosen from linear, saturated or unsaturated and/or branched and/or cyclic, aromatic or non-aromatic C₁ to C₃₀ alkylene groups, optionally including one or more heteroatoms,
X is a group defined by N—R₁₀R₁₁, or alternatively X constitutes an aromatic or non-aromatic ring, comprising a cationizable tertiary amine group, included in the ring or as a substituent, or may represent an aromatic or non-aromatic heterocycle containing a cationizable tertiary nitrogen included in the ring or as a substituent, or alternatively X takes one of the following meanings: guanidino, amidino or phosphino, and
R₁₀ and R₁₁ are chosen, independently, from a hydrogen atom and linear and/or branched and/or cyclic, aromatic or non-aromatic C₁ to C₁₆ alkyl groups, optionally. comprising heteroatoms, or alternatively R₁₀ and R₁₁ form, with the nitrogen to which they are attached, a fused or non-fused C₄ to C₃₀ heterocycle, optionally substituted with one or more identical or different radicals chosen from C₁ to C₄ alkyl groups, a hydroxyl group, a C₁ to C₄ alkoxy group and a halogen atom.

R₉ is preferably chosen from C₁ to C₃₀ alkyl, phenylene, benzylene, —(CH₂—CH═CH)— and —(CHOH)— groups.

Advantageously, R₉ is chosen from linear, branched or cyclic C₁ to C₆ alkylene groups, and phenylene and benzylene groups.

Preferably, R₁₀ and R₁₁ are chosen from a hydrogen atom and CH₃, C₂H₅, C₃H₇ and C₄H₉ groups.

Examples of heterocycles that are suitable for the meaning of X in its second alternative are pyridines, indolyl, isoindolinyl, imidazolyl, imidazolinyl, piperidyl, pyrazolinyl, pyrazolyl, quinoline, pyrazolinyl, pyridinyl, piperazinyl, pyrrolidinyl, quinidinyl, thiazolinyl, morpholine, and mixtures thereof

Examples of cationic hydrophilic monomers that may be mentioned include ethylenically unsaturated monomers comprising at least one primary, secondary or tertiary amine finction. Mention may be made especially of:
allylamine, allylpyridine;
aminoalkyl(meth)acrylates, such as [N,N-di(C₁-C₄)alkylamino](C₁-C₆)alkyl(meth)acrylates or [N-(C₁-C₄)alkylamino](C₁-C₆)alkyl(meth)acrylates and especially N,N dimethylaminoethyl(meth)acrylate, N,N-diethylaminoethyl(meth)acrylate, 2-aminoethyl(meth)acrylate and 2-(N-tert-butylamino)ethyl(meth)acrylate;
aminoalkyl(meth)acrylamides, such as (meth)acrylamides of [N,N-di(C₁-C₄)alkylamino](C₁-C₆)alkyl N,N-di(C₁-C₄)alkylamino)alkyl or [N-(C₁-C₄)alkylamino](C₁-C₆)alkyl(meth)acrylamides, and especially N,N-dimethylaminopropyl(meth)acrylamide, N,N-dimethylaminoethyl(meth)acrylamide and 3-aminopropyl(meth)acrylamide;
vinylamine, 2-(diethylamino)ethylstyrene;
N-vinylimidazole, N-vinyl-2-methylimidazole, N-vinylcarbazole;
and also mixtures thereof, and quatemized forms thereof.

Salts of mineral acids, such as sulphuric acid, hydrochloric acid, hydrobromic acid, hydriodic acid, phosphoric acid and boric acid, may be used to neutralize the cationic monomers.

Salts of organic acids, which may comprise one or more carboxylic, sulfonic or phosphonic acid groups, may also be mentioned. These may be linear, branched or cyclic aliphatic acids or alternatively aromatic acids. These acids may also comprise one or more heteroatoms chosen from O and N, for example in the form of hydroxyl groups. Mention may be made especially of propionic acid, acetic acid, terephthalic acid, citric acid and tartaric acid.

The tertiary amine groups may be quaternized:
with compounds containing labile halogen, especially alkyl halides such as C₁ to C₁₂ alkyl chlorides or bromides, for example methyl bromide or ethyl chloride,
compounds containing labile halogen, comprising carboxylic or sulfonic acid functions (optionally salified). Thus, amphoteric hydrophilic monomers (also known as betaines) are obtained.

The quaternizing agents may be, for example, sodium chloroacetate or cyclic sulfones, for example propane sulfone.

This quaternization and (or) salification reaction may take place on the already-synthesized polymer or on the starting monomer before performing the polymerization.

Examples of amphoteric monomers that may be mentioned include ethylenic carboxybetains or sulfobetains such as N,N-dimethyl-N-(2-methacryloyloxyethyl)-N-(3-sulfopropyl)ammonium betaine; N,N-dimethyl-N-(3-methacrylamidopropyl)-N-(3-sulfopropyl)ammonium betaine and 1-(3-sulfopropyl)-2-vinylpyridinium betaine.

The preferred cationic hydrophilic monomer is N,N′-dimethylaminoethyl(meth)acrylate.

The preferred hydrophilic monomer is (meth)acrylic acid.

Preferred Copolymers

According to one particularly preferred embodiment, the copolymer is chosen from:
methyl methacrylate/methacrylic acid copolymers; methyl methacrylate/acrylic acid copolymers, the said copolymers containing between 70% and 90% by weight of methyl methacrylate;
ethyl methacrylate/methacrylic acid copolymers; ethyl methacrylate/acrylic acid copolymers, the said copolymers containing between 70% and 90% by weight of ethyl methacrylate;
isobutyl methacrylate/methacrylic acid copolymers; isobutyl methacrylate/acrylic acid copolymers, the said copolymers containing between 70% and 90% by weight of isobutyl methacrylate;
benzyl methacrylate/methacrylic acid copolymers; benzyl methacrylate/acrylic acid copolymers, the said copolymers containing between 70% and 90% by weight of benzyl methacrylate;
benzyl acrylate/methacrylic acid copolymers; benzyl acrylate/acrylic acid copolymers, the said copolymers containing between 70% and 90% by weight of benzyl acrylate;
cyclohexyl methacrylate/methacrylic acid copolymers; cyclohexyl methacrylate/acrylic acid copolymers, the said copolymers containing between 70% and 90% by weight of cyclohexyl methacrylate;
cyclohexyl acrylate/methacrylic acid copolymers; cyclohexyl acrylate/acrylic acid copolymers, the said copolymers containing between 70% and 90% by weight of cyclohexyl acrylate;
tert-butyl methacrylate/methacrylic acid copolymers; tert-butyl methacrylate/acrylic acid copolymers, the said copolymers containing between 70% and 90% by weight of tert-butyl methacrylate;
tert-butyl acrylate/methacrylic acid copolymers; tert-butyl acrylate/acrylic acid copolymers, the said copolymers containing between 70% and 90% by weight of tert-butyl acrylate;
isobomyl methacrylate/methacrylic acid copolymers; isobomyl methacrylate/acrylic acid copolymers, the said copolymers containing between 70% and 90% by weight of isobomyl methacrylate;
isobomyl acrylate/methacrylic acid copolymers; isobomyl acrylate/acrylic acid copolymers, the said copolymers containing between 70% and 90% by weight of isobomyl acrylate;
norbomyl methacrylate/methacrylic acid copolymers; norbornyl methacrylate/acrylic acid copolymers, the said copolymers containing between 70% and 90% by weight of norbornyl.methacrylate;
norbomyl acrylate/methacrylic acid copolymers; norbornyl acrylate/acrylic acid copolymers, the said. copolymers containing between 70% and 90% by weight of norbornyl acrylate; and
styrene/methacrylic acid copolymers; styrene/acrylic acid copolymers, the said copolymers containing between 70% and 90% by weight of styrene.
   Monomer with a Tg <40° C.

According to one particular embodiment, the copolymers also comprising at least one “monomer unit” derived from a monomer whose corresponding homopolymer has a glass transition temperature of less than 40° C. (and especially greater than −100° C.) also form part of the invention provided that the overall glass transition temperature of the said copolymer remains. very much higher than or equal to 45° C.

According to another particular embodiment, the additional monomers whose homopolymers have a glass transition temperature of less than 40° C., which are known as “monomers with a Tg <40° C.”, are different from the hydrophobic monomers and the ionic hydrophilic monomers described above.

The content of monomers whose homopolymer has a glass transition temperature of less than 40° C. is adjusted such that the tensioning effect of the copolymer is not affected.

Their content in the copolymer is advantageously less than or equal to 25% by weight (especially from 0 to 25% by weight and preferably from 5% to 25% by weight) and even more preferably less than or equal to 10% by weight (especially from 0 to 10% by weight and preferably from 5% to 10% by weight), relative to the total weight of the copolymer.

The monomers whose homopolymers have glass transition temperatures of less than 40° C. are chosen from the following monomers:
C₂ to C₁₀ ethylenic hydrocarbons, such as ethylene, isoprene and butadiene,
the acrylates of formula:
   CH₂═CHCOOR₁₂
   in which R₁₂ represents:
   a linear or branched C₁ to C₁₂ alkyl group (with the exception of a tert-butyl group), in which is (are) optionally intercalated one or more identical or different heteroatoms, the said alkyl group also possibly being substituted with one or more identical or different substituents chosen from hydroxyl groups and halogen atoms (especially fluorine), preferably a linear or branched C₁ to C₁₂ alkyl group (with the exception of a tert-butyl group); examples of groups R₁₂ are methyl, ethyl, propyl, butyl, isobutyl, hexyl, ethylhexyl, octyl, lauryl, isooctyl, isodecyl, hydroxyethyl, hydroxypropyl, methoxyethyl, ethoxyethyl and methoxypropyl groups;
   a C₁ to C₁₂ alkyl-POE (polyoxyethylene) group, with repetition of the oxyethylene unit from 5 to 30 times, for example methoxy POE, R₁₂ then possibly being represented by R₁₃—(OC₂H₄)ₙ—, in which R₁₃ is a C₁ to C₁₂ alkyl group and n is an integer ranging from 5 to 30; or
   a polyoxyethylene group of structure —(CH₂CH₂O)ₙ—H in which n is an integer ranging from 5 to 30.
the methacrylates of formula:
   CH₂═C(CH₃)COOR₁₄
   in which R₁₄ represents:
   a linear C4 to C12 alkyl group or a branched C5 to C12 alkyl group (with the exception of cycloalkyl, aryl, aralkyl, heterocycloalkyl and heteroaralkyl groups), in which is (are) optionally intercalated one or more identical or different heteroatoms, the said alkyl group also possibly being substituted with one or more identical or different substituents chosen from hydroxyl groups and halogen atoms; and preferably a linear C4 to C12 alkyl group or a branched C5 to C12 alkyl group, which is unsubstituted; examples of groups R14 are hexyl, ethylhexyl, octyl, lauryl, isooctyl, isodecyl, dodecyl, methoxyethyl, methoxypropyl and ethoxyethyl groups,
   a C₁ to C12 alkyl-POE (polyoxyethylene) group, with repetition of the oxyethylene unit from 5 to 30 times, for example methoxy POE, R14 then possibly being represented by R13-(OC2H4)n-, in which R13 is a C₁ to C12 alkyl group and n is an integer ranging from 5 to 30; or
   a polyoxyethylene group of structure —(CH2CH2O)n-H in which n is an integer ranging from 5 to 30.

The particularly preferred monomers are: methyl acrylate, ethyl acrylate, propyl acrylate, n-butyl acrylate, 2-ethylhexyl acrylate, isobutyl acrylate, methoxyethyl acrylate, ethoxyethyl (meth)acrylate, n-hexyl(meth)acrylate.
The vinyl esters of formula:
   R₁₅COOCH═CH₂
   in which R₁₅ represents a linear or branched C₂ to C₁₂ alkyl group.

Examples of such vinyl esters are: vinyl propionate, vinyl butyrate, vinyl ethylhexanoate, vinyl neononanoate and vinyl neododecanoate.
Ethers of vinyl alcohol and of a C₁ to C₁₂ alcohol, such as methyl vinyl ether and ethyl vinyl ether.

The additional monomer is preferably chosen from the acrylates and methacrylates described above, and preferably from C₁-C₁₂ alkyl acrylates and C₄-C₁₂ alkyl methacrylates.

More particularly, the preferred additional monomers are:
the acrylates of formula:
   CH₂═CHCOOR₁₂
   in which R₁₂ represents a linear or branched C₁ to C₁₂ alkyl group (with the exception of a tert-butyl group), in which is (are) optionally intercalated one or more identical or different heteroatom(s), the said alkyl group also possibly being substituted with one or more identical or different substituents chosen from hydroxyl groups and halogen atoms, and
the methacrylates of formula:
   CH₂═C(CH₃)COOR₁₄
   in which R₁₄ represents a linear C₄ to C₁₂ alkyl group or a branched C₅ to C₁₂ alkyl group.
   Composition

The content, expressed as dry matter, of copolymer(s) present in a composition according to the present invention may range from 0.1% to 15%, preferably from 1% to 10% and even more preferably from 1% to 5% by weight relative to the total weight of the composition.

The composition of the invention may be in any galenical form normally used for topical application to the skin, especially in the form of an aqueous, aqueous-alcoholic or oily solution, an oil-in-water emulsion, an aqueous or oily gel, a liquid, pasty or solid anhydrous product, a dispersion of oil in an aqueous phase in the presence of spherules, these spherules possibly being polymeric nanoparticles such as nanospheres and nanocapsules or, better still, lipid vesicules of ionic and/or nonionic type.

Given that it is preferred to formulate the copolymer as defined in the context of the present invention in a form dispersed in water, compositions comprising an aqueous phase are preferred.

This aqueous phase may typically be present in a content of greater than or equal to 10%, preferably 20% and even more preferably 30%, or up to 50% by weight relative to the total weight of the composition.

This composition may be more or less fluid and may have the appearance of a white or coloured cream, a pomade, a milk, a lotion, a serum, a paste or a mousse. It may optionally be applied to the skin in aerosol form. It may also be in solid form, for example in the form of a stick. It may be used as a care product and/or as a makeup product for the skin. It may be used, for example, as a complexion product such as a foundation.

The composition of the invention more particularly constitutes an anti-ageing and in particular anti-wrinkle composition, which is especially in the form of a serum, i.e. a gelled aqueous composition. However, as a variant, it may constitute a bodycare composition and in particular a slimming composition.

In a known manner, the composition of the invention may also contain adjuvants that are common in cosmetics, such as hydrophilic or lipophilic gelling agents, hydrophilic or lipophilic active agents, preserving agents, antioxidants, solvents, fragrances, fillers, film-forming polymers, screening. agents, pigments, odour absorbers and dyestuffs. The amounts of these various adjuvants are those conventionally used in the field under consideration, for example from 0.01% to 20% of the total weight of the composition. Depending on their nature, these adjuvants may be introduced into the fatty phase, into the aqueous phase, into lipid vesicles and/or into nanoparticles. These adjuvants and the concentrations thereof should be such that they do not modify the desired tensioning property of the copolymer.

When the composition of the invention is an emulsion, the proportion of the fatty phase may range from 5% to 80% by weight and preferably from 5% to 50% by weight relative to the total weight of the composition. The fatty substances, emulsifiers and co-emulsifiers used in the composition in emulsion form are chosen from those conventionally used in the field under consideration. The emulsifier and the co-emulsifier are preferably present in the composition in a proportion ranging from 0.3% to 30% by weight and preferably from 0.5% to 20% by weight relative to the total weight of the composition.

As fatty substances that may be used in the invention, mention may be made of oils and especially mineral oils (liquid petroleum jelly), oils of plant origin (avocado oil or soybean oil), oils of animal origin (lanolin), synthetic oils (perhydrosqualene), silicone oils (cyclomethicone) and fluoro oils (perfluoropolyethers). Fatty alcohols (cetyl alcohol), fatty acids, waxes and gums, and in particular silicone gums, may also be used as fatty substances.

As emulsifiers and co-emulsifiers that may be used in the invention, examples that may be mentioned include fatty acid esters of polyethylene glycol, such as PEG-50 stearate and PEG-40 stearate, and fatty acid esters of polyols, such as glyceryl stearate and sorbitan tristearate.

Hydrophilic gelling agents that may be mentioned in particular include carboxyvinyl polymers (carbomer), acrylic copolymers such as acrylatelalkylacrylate copolymers, polyacrylamides, polysaccharides, natural gums and clays, and lipophilic gelling agents that may be mentioned include modified clays, for instance bentones, the metal salts of fatty acids, hydrophobic silica and polyethylenes.

In addition, other compounds known to those skilled in the art as tensioning agents and having properties different from those of the agents used according to the invention may also be combined with the tensioning agents used according to the invention, especially synthetic latices, plant proteins, polysaccharides of plant origin optionally in the form of microgels, starches, mixed silicates and colloidal particles of mineral fillers.

As a variant or in addition, when the composition according to the invention is a slimming composition, it is preferable for it to comprise, in a physiologically acceptable medium, at least one copolymer as defined above and one or more draining, lipolytic, de-infiltrating, slimming, firming, anti-glycating and/or vaso-protective compounds.

The amount of slimming active agent(s) may vary within a wide range and depends on the nature of the active agent(s) used. In general, the slimming active agent(s) is (are) present in a concentration ranging from 0.05% to 20% and preferably from 0.1% to 10% by weight relative to the total weight of the composition.

When the composition according to the invention constitutes an anti-ageing composition, it will be advantageous to introduce into this composition at least one compound chosen from: desquamating agents; moisturizers; depigmenting or pro-pigmenting agents; anti-glycation agents; NO-synthase inhibitors; agents for stimulating the synthesis of dermal or epidermal macromolecules and/or for preventing their degradation; agents for stimulating the proliferation of fibroblasts and/or keratinocytes or for stimulating keratinocyte differentiation; other muscle relaxants and/or dermo-decontracting agents; tensioning agents; anti-pollution agents and/or free-radical scavengers; agents acting on the capillary circulation; agents acting on the energy metabolism of cells; and mixtures thereof.

Examples of such additional compounds are: retinol and its derivatives such as retinyl palmitate; ascorbic acid and its derivatives such as magnesium ascorbyl phosphate and ascorbyl glucoside; tocopherol and its derivatives such as tocopheryl acetate; nicotinic acid and its precursors such as nicotinamide; ubiquinone; glutathione and its precursors such as L-2-oxothiazolidine-4-carboxylic acid; plant extracts and especially plant proteins and hydrolysates thereof, and also plant hormones; marine extracts such as algal extracts; bacterial extracts; sapogenins such as diosgenin and wild yam extracts containing them; ceramides; hydroxy acids, such as salicylic acid and 5-n-octanoylsalicylic acid; resveratrol; oligopeptides and pseudodipeptides and acyl derivatives thereof; manganese and magnesium salts, in particular the gluconates; and mixtures thereof.

The amounts of these active agents may vary within a wide range. In general, these active agents are present in a concentration ranging from 0.01% to 15% and preferably from 0.05% to 10% by weight relative to the total weight of the composition.

In the event of incompatibility, the active agents indicated above may be incorporated into spherules, especially ionic or nonionic vesicles and/or nanoparticles (nanocapsules and/or nanospheres), so as to isolate them from each other in the composition.

The examples below of compositions according to the invention are given as illustrations and with no limiting nature. The amounts therein are given as weight percentages. The degrees of neutralization of the copolymers are 100%.

In the examples that follow, “Mp” is the abbreviation for the peak molecular mass; “Mn” for the number-average molecular mass, “Mw” for the weight-average molecular mass and “Ip” for the polydispersity index.

### EXAMPLES

### Example 1

### Example of Synthesis of a Methyl Methacrylate/Methacrylic Acid Statistical Copolymer

1st Step: Synthesis of the Polymer

1 g of Trigonox 21S (t-butylperoxy 2-ethylhexanoate) and 200 g of methyl ethyl ketone are placed in a 21 jacketed reactor. The mixture is refluxed for one hour. After one hour, a mixture of 170 g of methyl methacrylate and 30 g of methacrylic acid is added dropwise over a period of one hour. The colourless mixture becomes viscous. Heating is stopped six hours after addition of the monomers.

Composition by NMR: 85.1% methyl methacrylate, 14.9% methacrylic acid

Mass by GPC in tetrahydrofuran (THF) (polystyrene standards): Mp=98,772 g.mol⁻¹; Mn=61,261 g.mol⁻¹; Mw=105,698 g.mol⁻¹ Ip=1.7

2nd Step: Dispersion of the Polymer in Water

200 g of methyl ethyl ketone are added to the above reaction medium and the mixture is heated to 60° C. 30.86 g of 2-amino-2-methylpropanol are added dropwise to neutralize the acid finctions, and 1200 g of water are added. The volatile solvents are evaporated off by heating to 100° C. A transparent yellow aqueous dispersion is obtained.

### Example 2

### Example of Synthesis of a Cyclohexyl Methacrylate/Methacrylic Acid Statistical Copolymer

1st step: Synthesis of the Polymer

260 g of methyl ethyl ketone are placed in a three-necked flask equipped with a magnetic bar, followed by the monomers, 42.5 g of cyclohexyl methacrylate and 7.5 g of methacrylic acid. A stream of nitrogen is circulated in the flask for a few minutes so as to remove the ambient oxygen. Finally, the initiator is introduced, 155 μL of Trigonox 21S (t-butylperoxy 2-ethylhexanoate). The reaction medium is then heated in an oil bath at 80° C. for 8 hours. A viscous product is obtained.

The polymer is precipitated by adding the reaction medium dropwise to 4 l of methanol with stirring. The polymer is recovered by filtration and then dried under vacuum. 83% yield.

Composition by NMR: 83.7% cyclohexyl methacrylate, 16.3% methacrylic acid

Mass by GPC in THF (polystyrene standards): Mp=134,315 g.mol⁻¹; Mn=85,905 g.mol⁻¹; Mw=159,093 g.mol⁻¹ Ip=1.85

2nd step: Dispersion of the Polymer in Water

40 g of the above polymer are dissolved in 220 g of tetrahydrofuran. The acid functions are neutralized by adding 6.75 g of 2-amino-2-methylpropanol. 160 g of water are then added to the medium, and the tetrahydrofuran is evaporated off under reduced pressure on a rotary evaporator. After evaporation, an opalescent dispersion at 22% by mass in water is obtained.

### Example 3

### Example of Synthesis of a Methyl Methacrylate/Methyl Acrylate/Methacrllic Acid Statistical Copolymer

1st Step: Synthesis of the Polymer

50 g of methyl ethyl ketone are placed in a three-necked flask equipped with a magnetic bar, followed by the monomers, 35 g of methyl methacrylate, 7.5 g of methyl acrylate and 7.5 g of methacrylic acid. A stream of nitrogen is circulated in the flask for a few minutes in order to remove the ambient oxygen. Finally, the initiator is introduced, 143 μL of Trigonox 21S (t-butylperoxy-2-ethylhexanoate). The reaction medium is then heated in an oil bath at 80° C. for 8 hours. A viscous product is obtained.

The polymer is precipitated by adding the reaction medium dropwise to 2 l of an ethanol/water mixture (50/50) with stirring. The polymer is recovered by filtration and then dried under vacuum. 54% yield.

Composition by NMR: 71% methyl methacrylate, 14% methyl acrylate; 16% methacrylic acid

Mass by GPC in THF (polystyrene standards): Mp=113,249 g.mol⁻¹; Mn=71,648 g.mol⁻¹; Mw=105,358 g.mol⁻¹ Ip=1.47

2nd Step: Dispersion of the Polymer in Water

26.8 g of the above polymer are dissolved in 110 g of tetrahydrofuran and 10 g of EtOH. The mixture is heated at 60° C. for 6 hours to complete the dissolution, and is then cooled to room temperature. The acid functions are neutralized by adding 4.16 g of 2-amino-2-methylpropanol. 150 g of water are then added to the medium and the tetrahydrofuran and ethanol are evaporated off under reduced pressure on a rotary evaporator. After evaporation, a cohesive opalescent gel at 12.5% by mass in water is obtained.

### Example 4

### Example of Synthesis of a Methyl Methacrylate/Ethyl Acrylate/Methacrylic Acid Statistical Copolymer

1st Step: Synthesis of the Polymer

50 g of methyl ethyl ketone are placed in a three-necked flask equipped with a magnetic bar, followed by the monomers, 37.5 g of methyl methacrylate, 5 g of ethyl acrylate and 7.5 g of methacrylic acid. A stream of nitrogen is circulated in the flask for a few minutes so as to remove the ambient oxygen. Finally, the initiator is introduced, 143 μL of Trigonox 21S (t-butylperoxy 2-ethylhexanoate). The reaction medium is then heated in an oil bath at 80° C. for 8 hours. A viscous product is obtained.

The polymer is precipitated by adding the reaction medium dropwise to 2 l of an ethanol/water mixture (50/50) with stirring. The polymer is recovered by filtration and then dried under vacuum. 92% yield.

Composition by NMR: 75.7% methyl methacrylate, 6.7% ethyl acrylate; 15% methacrylic acid

Mass by GPC in THF (polystyrene standards): Mp=115,523 g.mol⁻¹; Mn=63,149 g.mol⁻¹; Mw=124,703 g.mol⁻¹ Ip=1.97

2nd Step: Dispersion of the Polymer in Water

37.5 g of the above polymer are dissolved in 150 g of tetrahydrofuran and 10 g of EtOH. The mixture is heated at 60° C. for 6 hours to complete the dissolution, and is then allowed to return to room temperature. The acid functions are neutralized by adding 5.8 g of 2-amino-2-methylpropanol. 210 g of water are then added to the medium, and the tetrahydrofuran and ethanol are evaporated off under reduced pressure on a rotary evaporator. After evaporation, a viscous transparent dispersion at 13.6% by mass in water is obtained.

### Example 5

### Example of Synthesis of a Methyl Methacrylate/Methacrylic Acid Statistical Copolymer

1st Step: Synthesis of the Polymer

4.8 g of Trigonox 21S (t-butylperoxy 2-ethylhexanoate) and 360 g of methyl ethyl ketone are placed in a 2 l jacketed reactor. The mixture is refluxed for one hour. After one hour, a mixture of 102 g of methyl methacrylate and 18 g of methacrylic acid is added dropwise over a period of one hour. The colourless mixture becomes viscous. The heating is stopped six hours after adding the monomers.

Composition by NMR: 86.7% methyl methacrylate, 13.3% methacrylic acid Mass by GPC in tetrahydrofuran (THF) (polystyrene standards): Mp=25,200 g.mol⁻¹; Mn=13,900 g.mol⁻¹; Mw=30,450 g.mol⁻¹ Ip=2.19

2nd Step: Dispersion of the Polymer in Water

16.5 g of 2-amino-2-methylpropanol are added dropwise, at room temperature, to the above reaction medium to neutralize the acid functions, and 360 g of water are added. The volatile solvents are evaporated off by heating to 100° C. A transparent yellow aqueous dispersion with a solids content of 26.9% is obtained

### Example 6 (Comparative)

### Example of Synthesis of a Methyl Methacrylate/Methacrylic Acid Statistical Copolymer

1st Step: Synthesis of the Polymer

300 g of ethanol are placed in a three-necked flask equipped with a magnetic bar, followed by the monomers, 50 g of methyl methacrylate and 50 g of methacrylic acid. A stream of nitrogen is circulated in the flask for a few minutes so as to remove the ambient oxygen. Finally, the initiator is introduced, 0.5 g of Trigonox 21S (t-butylperoxy 2-ethylhexanoate). The reaction medium is then heated in an oil bath at 90° C. for 8 hours. A viscous product is obtained.

The reaction medium is diluted with 150 g of ethanol and is precipitated by dropwise addition to 2 l of water with stirring. The polymer is recovered by filtration and then dried under vacuum. The yield is 80%.

Composition by NMR: 65.6% methyl methacrylate, 34.4% methacrylic acid

2nd Step: Dispersion of the Polymer in Water

50.6 g of the above polymer are dissolved in 204 g of tetrahydrofuran. The acid functions are neutralized by adding 18 g of 2-amino-2-methylpropanol. 184 g of water are then added to the medium and the tetrahydrofuran is evaporated off under reduced pressure on a rotary evaporator. After evaporation, an opaque dispersion at a concentration of 17% by mass in water is obtained.

### Example 7 (Comparative)

### Example of Synthesis of a Methyl Methacrylate/Methacrylic Acid Statistical Copolymer

1st Step: Synthesis of the Polymer

8 g of Trigonox 21S (t-butylperoxy 2-ethylhexanoate) and 300 g of methyl ethyl ketone are placed in a 2 l jacketed reactor. The mixture is refluxed for one hour. After one hour, a mixture of 85 g of methyl methacrylate and 15 g of methacrylic acid is added dropwise over a period of one hour. The colourless mixture becomes viscous. The heating is stopped six hours after the addition of the monomers.

Composition by NMR: 80.3% methyl methacrylate, 19.7% methacrylic acid

Mass by GPC in tetrahydrofuran (THF) (polystyrene standards): Mp=11,100 g.mol⁻¹; Mn=6,600 g.mol⁻¹; Mw=14,600 g.mol⁻¹ Ip=2.21

2nd Step: Dispersion of the Polymer in Water

20.4 g of 2-amino-2-methylpropanol are added dropwise, at room temperature, to the above reaction medium to neutralize the acid finctions, and 300 g of water are added. The volatile solvents are evaporated off by heating to 100° C. A transparent yellow aqueous dispersion with a solids content of 29.6% is obtained.

### Example 8

### Anti-wrinkle Serum

The composition below was prepared:

A

| | |
|---|---|
| Water | 50.45 g |
| Synthetic thickening polymer | 2.00 g |
| Preserving agents | 0.85 g |

B

| | |
|---|---|
| Copolymer of Example 1 (15% dispersion in water) | 46.70 g |

Procedure:

Phase A is heated to about 75° C., with stirring, the heating is stopped, while continuing the stirring, down to room temperature and phase B is then added. Gentle stirring is then continued for 30 minutes.

### Example 9

### Anti-wrinkle Cream (O/W Emulsion)

The composition below was prepared:

A

| | | |
|---|---|---|
| Glyceryl stearate (and) PEG-100 stearate | 2.00 | g |
| Dimyristyl tartrate (and) cetearyl alcohol (and) | 1.50 | |
| C12-15 pareth-7 (and) PPG-25 laureth-25 | | |
| Cyclohexasiloxane | 10.00 | |
| Stearyl alcohol | 1.00 | g |

B

| | | |
|---|---|---|
| Water | 37.15 | g |
| Phenoxyethanol | 1.00 | g |
| Pentasodium ethylenediaminetetramethylene phosphate | 0.05 | g |
| Ammonium polyacryldimethyltauramide (thickener) | 0.40 | g |
| Xanthan gum | 0.20 | g |

C

| | | |
|---|---|---|
| Copolymer of Example 1 (15% dispersion in water) | 46.70 | g |

Procedure:

The constituents of phase B except for the thickener are heated to about 75° C., and the thickener is then incorporated therein; the resulting mixture is stirred until a uniform gel is obtained.

Phase A is heated to about 75° C. and the emulsion is then prepared by incorporating phase A into phase B.

Finally, at 40-45° C., phase C is incorporated and stirring is continued until cooling is complete.

The serum, applied to the face, allows wrinkles to be smoothed out or even effaced.

Similar compositions were also prepared by using, respectively, the polymer of Examples 3, 4 and 5. The compositions obtained, applied to the face, allow wrinkles to be smoothed out efficiently.

### Example 10

### Demonstration of the Tensioning Effect of Polymers According to the Invention

10-1 in vitro Quantification of the Tensioning Effect of the Copolymers

It has been shown that the tensioning power could be described by means of an in vitro retraction test. This test consists in quantifying in vitro the tensioning power of a material placed on an elastomeric substrate with a modulus of about 20 MPa and a thickness of 100 μm. The solution containing the tensioning agent at 7% by weight is thus deposited (30 μl) onto a rectangular (10×40 mm) specimen of elastomer. After drying for 3-4 hours at 22±3° C. and 40±10% RH, the tension exerted by this deposit on the substrate, consequently the tensioning power, is directly related to the decrease in width at the centre of the specimen. The tensioning effect (TE) may thus be quantified in the following manner:
“TE”=(L_{0-L}₃ₕ/L₀)×100 in %
with L₀=initial width 10 mm
and L₃ₕ=width after drying for 3 hours

The result presented below shows, with regard to the desired cosmetic application, a very large tensioning effect obtained with the copolymers in accordance with the invention, even in the presence of glycerol.

| | TE (%) |
|---|---|
| Copolymer of Example 1 (7% in H₂O) | 83 ± 16 |
| Copolymer of Example 1 (7% in H₂O) + 3% glycerin | 65 ± 15 |
| Copolymer of Example 2 (7% in H₂O) | 80 ± 8 |
| Copolymer of Example 2 (7% in H₂O) + 3% glycerin | 85 ± 12 |
| Copolymer of Example 3 (7% in H₂O) | 100 ± 5 |
| Copolymer of Example 4 (7% in H₂O) | 95 ± 9 |
| Copolymer of Example 5 (7% in H₂O) | 71 ± 4 |
| Copolymer of Comparative Example 6 (7% in H₂O) | 15 ± 6 |
| Copolymer of Comparative Example 6 (7% in H₂O) + | 0 |
| 3% glycerin | |
| Copolymer of Comparative Example 7 (7% in H₂O) | 29 ± 3 |

10-2 in vivo Evaluation on Women with Mature Skin (Cabin Test) of the Anti-wrinkle Effect of Cosmetic Compositions Containing the Copolymer of Example 1:

The composition corresponding to Example 8 was tested on a panel of six 40- to 60-year-old women with wrinkles and fine lines around the contour of the eyes. After applying these formulations, a substantial mechanical smoothing effect on the wrinkles and fine lines was observed on all the models.

0-3 Determination of the Glass Transition Temperatures of the Copolymers:

The glass transition temperatures were determined by DSC according to the protocol reported in the description.

| | Tg (° C.) |
|---|---|
| Copolymer of Example 1 | 81° C. |
| Copolymer of Example 2 | 88° C. |
| Copolymer of Comparative Example 6 | 40° C. |

The glass transition temperatures of the copolymers of Examples 3, 4, 5 and 7 are greater than 45° C.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be realised without departing from the spirit and scope of the present invention.

What is claimed is:

1. Cosmetic composition comprising, in a physiologically acceptable medium, at least one statistical copolymer with a linear main chain of ethylenic nature, in which said copolymer:
(i) has a weight-average molecular mass of between 15,000 and 600,000 g/mol, and consists of:
(a) at least 70% by weight of monomer units derived from monomers for which the homopolymers are hydrophobic and have a glass transition temperature of greater than 40° C.,
(b) at least one monomer unit derived from an at least partially neutralized ionic hydrophilic monomer, and
(c) 0 to 25% by weight of monomer units derived from additional monomers for which the homopolymers have a glass transition temperature of less than 40° C., selected from the group consisting of:
acrylates of formula:
CH₂═CHCOOR₁₂
in which R₁₂ represents a linear or branched C₁ to C₁₂ alkyl group with the exception of a tert-butyl group, in which is (are) optionally intercalated one or more. identical or different heteroatoms, said alkyl group also optionally being substituted with one or more identical or different substituents selected from the group consisting of hydroxyl groups and halogen atoms, and
methacrylates of formula:
CH₂═C(CH₃)COOR₁₄
in which R₁₄ represents a linear C₄ to C₁₂ alkyl group or a branched C₅ to C₁₂ alkyl group, and
(ii) has an overall glass transition temperature of greater than or equal to 45° C.

2. Cosmetic composition according to claim 1, in which the copolymer is in dispersion in water.

3. Cosmetic composition according to claim 1, in which the copolymer has more than 75%, relative to its total weight, of monomer units derived from monomers whose homopolymers have a glass transition temperature of greater than 40° C.

4. Cosmetic composition according to claim 1, in which the copolymer has more than 80% by weight relative to its total weight, of “monomer units” derived from monomers whose homopolymers have a glass transition temperature of greater than 40° C.

5. Cosmetic composition according to claim 1, in which the copolymer has up to 95% by weight relative to its total weight, of “monomer units” derived from monomers whose homopolymers have a glass transition temperature of greater than 40° C.

6. Cosmetic composition according to claim 1, in which the monomers whose homopolymers have a glass transition temperature of greater than 40° C. are selected from the group consisting of vinyl compounds, acrylates, methacrylates and (meth)acrylamides.

7. Cosmetic composition according to claim 1, in which the monomers whose homopolymers have a glass transition temperature of greater than 40° C. are selected from the group consisting of:
vinyl compounds of formula:
CH₂═CHR₁,
in which R₁ is a group 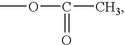
a C₃ to C₈ cycloalkyl group or a C₆ to C₂₀ aryl group,
acrylates of formula:
CH₂═CHCOOR₂
in which R₂ is a tert-butyl group, a C₃ to C₈ cycloalkyl group optionally bridged with a C₁ to C₄ alkylene group, or a C₇ to C₃₀ aralkyl group having a C₁ to C₄ alkyl group,
methacrylates of formula:
CH₂═C(CH₃)COOR₃
in which R₃ is an isobutyl or tert-butyl group, a linear or branched C₁ to C₃ alkyl group, a C₃ to C₈ cycloalkyl group optionally bridged with a C₁ to C₄ alkylene group, or a C₇ to C₃₀ aralkyl group having a C₁ to C₄ alkyl group, and
(meth)acrylamides of formula: 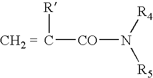
in which R′ denotes H or —CH₃, and in which R₄ and R₅, which may be identical or different, each represent a hydrogen atom or a linear or branched C₄ to C₁₂ alkyl group, wherein R₄ and R₅ cannot simultaneously represent a hydrogen atom.

8. Cosmetic composition according to claim 1, in which the monomers whose homopolymers have a glass transition temperature of greater than 40° C. are selected from the group consisting of styrene, benzyl acrylate, C₃ to C₈ cycloalkyl acrylate optionally bridged with a C₁ to C₄ alkylene group, tert-butyl acrylate, C₁ to C₃ alkyl methacrylate, tert-butyl methacrylate, benzyl methacrylate and C₃ to C₈ cycloalkyl methacrylate optionally bridged with a C₁ to C₄ alkylene group.

9. Cosmetic composition according to claim 1, in which the monomers whose homopolymers have a glass transition temperature of greater than 40° C. are selected from the group consisting of vinylcyclohexane, styrene, vinyl acetate, benzyl acrylate, cyclohexyl acrylate, tert-butyl acrylate, isobomyl acrylate, norbomyl acrylate, methyl, ethyl, isobutyl, cyclohexyl, benzyl, tert-butyl, isobomyl and norbomyl methacrylate, N-butylacrylamide, N-t-butylacrylamide and N,N-dibutylacrylamide.

10. Cosmetic composition according to claim 1, in which the monomers whose homopolymers have a glass transition temperature of greater than 40° C. are selected from the group consisting of benzyl acrylate, cyclohexyl acrylate, tert-butyl acrylate, isobomyl acrylate, norbomyl acrylate, methyl, ethyl, isobutyl, cyclohexyl, benzyl, tert-butyl, isobomyl and norbomyl methacrylate and styrene.

11. Cosmetic composition according to claim 1, in which the monomers whose homopolymers have a glass transition temperature of greater than 40° C. are selected from the group consisting of methyl methacrylate and cyclohexyl methacrylate.

12. Cosmetic composition according to claim 1, in which the ionic hydrophilic monomer is selected from the group consisting of anionic hydrophilic monomers, cationic hydrophilic monomers, amphoteric monomers, and mixtures thereof.

13. Cosmetic composition according to claim 12, in which the ionic hydrophilic monomer includes an anionic hydrophilic monomer defined by formula (I):
CH₂═CR₆ (Z)ₙ (R₇)ₘ Y (I)
in which Z has one of the following meanings: C(═O)O, C(═O)NH, O, O(C═O),
n is equal to 0 or 1,
m is equal to 0 or 1,
R₆ is a hydrogen atom, a CH₃ group or a C₂ to C₃₀ alkyl group,
R₇ is selected from the group consisting of linear, saturated or unsaturated and/or branched and/or cyclic (aromatic or non-aromatic) C₁ to C₃₀ alkylene groups, optionally including one or more heteroatoms, and
Y is selected from the following groups: —COOH, —SO₃H, —OSO₃H, —OP(OH)₂, and —OPO(OH)₂.

14. Cosmetic composition according to claim 13, in which n is equal to 0 in formula (I).

15. Cosmetic composition according to claim 13, in which m is equal to 0 in formula (I).

16. Cosmetic composition according to claim 13, in which R₆ is a hydrogen atom or a CH₃ or C₂H₅ group and R₇ is selected from the group consisting of C₁ to C₃₀ alkylene, phenylene, benzylene, —(CH₂—CH═CH)— and (CHOH) groups.

17. Cosmetic composition according to claim 16, in which R₆ is a CH₃ group.

18. Cosmetic composition according to claim 12, in which the ionic hydrophilic monomer includes an anionic hydrophilic monomer selected from the group consisting of:
ethylenically unsaturated monomers comprising at least one carboxylic acid (COOH), phosphonic acid (PO₃H₂) or sulfonic acid (SO₃H) function,
carboxylic anhydrides bearing a vinyl bond,
diacids,
and mixtures thereof.

19. Cosmetic composition according to claim 1, in which the hydrophilic monomer is (meth)acrylic acid.

20. Cosmetic composition according to claim 1, in which the weight content of monomer units derived from ionic hydrophilic monomers in the copolymer is between 5% and 30% relative to the total weight of the copolymer.

21. Cosmetic composition according to claim 1, in which the weight content of monomer units derived from ionic hydrophilic monomers in the copolymer is between 10% and 25% relative to the total weight of the copolymer.

22. Cosmetic composition according to claim 1, in which the weight content of monomer units derived from ionic hydrophilic monomers in the copolymer is between 10% and 20% relative to the total weight of the copolymer.

23. Cosmetic composition according to claim 1, in which the copolymer is selected from the group consisting of:
methyl methacrylate/methacrylic acid copolymers; methyl methacrylate/acrylic acid copolymers, said copolymers containing between 70% and 90% by weight of methyl methacrylate;
ethyl methacrylate/methacrylic acid copolymers; ethyl methacrylate/acrylic acid copolymers, said copolymers containing between 70% and 90% by weight of ethyl methacrylate;
isobutyl methacrylate/methacrylic acid copolymers; isobutyl methacrylate/acrylic acid copolymers, said copolymers containing between 70% and 90% by weight of isobutyl methacrylate;
benzyl methacrylate/methacrylic acid copolymers; benzyl methacrylate/acrylic acid copolymers, said copolymers containing between 70% and 90% by weight of benzyl methacrylate;
benzyl acrylate/methacrylic acid copolymers; benzyl acrylate/acrylic acid copolymers, said copolymers containing between 70% and 90% by weight of benzyl acrylate;
cyclohexyl methacrylate/methacrylic acid copolymers; cyclohexyl methacrylate/acrylic acid copolymers, said copolymers containing between 70% and 90% by weight of cyclohexyl methacrylate;
cyclohexyl acrylate/methacrylic acid copolymers; cyclohexyl acrylate/acrylic acid copolymers, said copolymers containing between 70% and 90% by weight of cyclohexyl acrylate;
tert-butyl methacrylate/methacrylic acid copolymers; tert-butyl methacrylate/acrylic acid copolymers, said copolymers containing between 70% and 90% by weight of tert-butyl methacrylate;
tert-butyl acrylate/methacrylic acid copolymers; tert-butyl acrylate/acrylic acid copolymers, said copolymers containing between 70% and 90% by weight of tert-butyl acrylate;
isobomyl methacrylate/methacrylic acid copolymers; isobomyl methacrylate/acrylic acid copolymers, said copolymers containing between 70% and 90% by weight of isobomyl methacrylate;
isobomyl acrylate/methacrylic acid copolymers; isobomyl acrylate/acrylic acid copolymers, said copolymers containing between 70% and 90% by weight of isobornyl acrylate;
norbornyl methacrylate/methacrylic acid copolymers; norbornyl methacrylate/acrylic acid copolymers, said copolymers containing between 70% and 90% by weight of norbornyl methacrylate;
norbornyl acrylate/methacrylic acid copolymers; norbornyl acrylate/acrylic acid copolymers, said copolymers containing between 70% and 90% by weight of norbornyl acrylate; and
styrene/methacrylic acid copolymers; styrene/acrylic acid copolymers, said copolymers containing between 70% and 90% by weight of styrene.

24. Cosmetic composition according to claim 1, in which the copolymer includes at least one said monomer unit derived from a monomer for which the homopolymer has a glass transition temperature of less than 40° C.

25. Cosmetic composition according to claim 1, in which the monomer for which the homopolymer has a glass transition temperature of less than 40° C. is present in a content ranging from 5% to 25% by weight relative to the total weight of the copolymer.

26. Cosmetic composition according to claim 1, in which the monomer for which the homopolymer has a glass transition temperature of less than 40° C. is present in a content of less than or equal to 10% by weight relative to the total weight of the copolymer.

27. Cosmetic composition according to claim 1, in which the monomer for which the homopolymer has a glass transition temperature of less than 40° C. is present in a content ranging from 0% to 10% by weight relative to the total weight of the copolymer.

28. Cosmetic composition according to claim 1, in which the monomer for which the homopolymer has a glass transition temperature of less than 40° C. is present in a content ranging from 5% to 10% by weight relative to the total weight of the copolymer.

29. Cosmetic composition according to claim 1, in which the copolymer is included in a content, expressed as dry matter, ranging from 0.1% to 15% by weight relative to the total weight of the composition.

30. Cosmetic composition according to claim 1, in which the copolymer is included in a content, expressed as dry matter, ranging from 1% to 10% by weight relative to the total weight of the composition.

31. Cosmetic composition according to claim 1, in which the copolymer is included in a content, expressed as dry matter, ranging from 1% to 5% by weight relative to the total weight of the composition.

32. Cosmetic composition according to claim 18, in which the ionic hydrophilic monomer includes an anionic hydrophilic monomer selected from the group consisting of acrylic acid, methacrylic acid, 2-carboxyethyl(meth)acrylate, vinylbenzoic acid, acrylamido-glycolic acid CH₂═CHCONHCH(OH)CO₂H, maleic acid, acrylamidopropanesulfonic acid, acrylamido-2-methylpropanesulfonic acid, styrenesulfonic acid, vinylsulfonic acid, propyl-3-sulfonic acid methacrylate or acrylate (CH₂═C(CH₃)CO₂(CH₂)₃SO₃H), ethyl-2-sulfonic acid methacrylate or acrylate (CH₂═C(CH₃)CO₂(CH₂)₂SO₃H) and methyl vinyl sulfone, vinylphosphonic acid (CH₂═CH—PO(OH)₂) and ethyl-2-phosphonic acid methacrylate (CH₂═C(CH₃)COOCH₂CH₂OP(O)(OH)₂), maleic anhydride, crotonic acid, itaconic acid, fumaric acid and maleic acid and mixtures thereof.

33. Cosmetic method for tensioning skin comprising applying to said skin a cosmetic composition containing a copolymer as described in claim 1.

34. Cosmetic method for reducing or treating age signs comprising applying to skin at least one composition comprising at least one statistical copolymer with a linear main chain of ethylenic nature in an amount that is effective for smoothing out or effacing wrinkles and fine lines on human skin by means of a tensioning effect, wherein said copolymer:
(i) has a weight-average molecular mass of between 15,000 and 600,000 g/mol, and consists of:
(a) at least 70% by weight of monomer units derived from monomers for which the homopolymers are hydrophobic and have a glass transition temperature of greater than 40° C.,
(b) at least one monomer unit derived from an at least partially neutralized ionic hydrophilic monomer, and
(c) 0 to 25% by weight of monomer units derived from additional monomers for which the homopolymers have a glass transition temperature of less than 40° C., selected from the group consisting of:
acrylates of formula:
CH₂═CHCOOR₁₂
in which R₁₂ represents a linear or branched C₁ to C₁₂ alkyl group with the exception of a tert-butyl group, in which is (are) optionally intercalated one or more identical or different heteroatoms, the alkyl group also optionally being substituted with one or more identical or different substituents selected from the group consisting of hydroxyl groups and halogen atoms, and
methacrylates of formula:
CH₂═C(CH₃)COOR₁₄
in which R₁₄ represents a linear C₄ to C₁₂ alkyl group or a branched C₅ to C₁₂ alkyl group, and
(ii) has an overall glass transition temperature of greater than or equal to 45° C.

35. Cosmetic method according to claim 34, comprising applying said composition to skin wrinkles.
